(19) United States
(12) Patent Application Publication
(10) Pub. No.: US 2009/0178672 A1
(43) Pub. Date: Jul. 16, 2009

(54) METHODS, SYSTEMS AND COMPUTER-READABLE PRODUCTS FOR OPTIMIZING AEROSOL PARTICLE ADMINISTRATION TO THE LUNGS

(75) Inventors: Bernhard Mullinger, Munich (DE); Philipp Kroneberg, Olching (DE); Marion Wencker, Frankfurt (DE)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: ACTIVAERO GMBH, Gemunden (DE)

(21) Appl. No.: 12/327,940

(22) Filed: Dec. 4, 2008

(30) Foreign Application Priority Data
Dec. 5, 2007 (EP) 07122405.9

(51) Int. Cl. A61M 11/00; A61B 5/087

(52) U.S. Cl. 128/200.14; 600/538

(57) ABSTRACT

The present invention is directed to methods, systems and computer-readable products for optimizing aerosol particle administration to the lungs. More specifically, the present invention relates to methods of tailoring inhaling maneuvers for aerosol particle administration using the subject's lung function data and to systems and computer-readable products carrying out such methods.

### FIELD OF THE INVENTION

The present invention is directed to methods, systems and computer-readable products for optimizing aerosol particle administration to the lungs. More specifically, the present invention relates to methods of tailoring inhaling maneuvers for aerosol particle administration using a subject's lung function data and to systems and computer-readable products carrying out such methods.

### BACKGROUND OF THE INVENTION

Of particular interest to the invention are pulmonary delivery techniques which involve the inhalation of a pharmaceutical formulation by a patient so that a drug or active agent within the formulation can reach the lungs. Pulmonary delivery techniques can be advantageous for certain respiratory diseases in that it allows selective delivery of optimal concentrations of pharmaceutical formulations to the airways while causing fewer side effects than systematic administration. Although pulmonary delivery techniques allow selective delivery, effective administration of inhaled formulations has been found to vary depending on the mode of inhalation, aerosol particle characteristics and characteristics of the patient. Since many parameters determining administration are patient-related, patients often receive training for the proper breathing maneuver which ensures accurate administration of the formulation. However, such training can be an inconvenience to the patient and/or the health professional. Also, many patients find it cumbersome to maintain the proper breathing maneuver over time and, thus, patient compliance has not been ideal. Therefore, it would be advantageous to optimize pulmonary delivery techniques by adjusting the aerosol particle administration parameters of the inhalation device to the patient's breathing capabilities, such that it would be no longer necessary for patients to concentrate on the proper breathing maneuvers. For example, US 2001/0037806A1, the contents of which are incorporated herein by reference, describes a device for the controlled inhalation of therapeutic aerosols.

### SUMMARY OF THE INVENTION

The present invention provides improved methods of optimizing aerosol particle administration by adapting aerosol parameters using a subject's lung function data.

The present invention also provides systems and computer-readable products for autonomously carrying out the methods of the present invention.

The present invention also provides an inhalation system having aerosol parameters that can be adapted automatically depending on measurement of at least one lung function parameter indicative of a subject's inhalation or exhalation function to thereby tailor aerosol particle administration to the subject's breathing capabilities.

The present invention comprises a method for determining aerosol particle administration to the lungs. The method comprises deriving aerosol parameters for an inhalation system based on at least one measured pulmonary function parameter to thereby adapt the aerosol particle administration to a subject's lung function. By adapting the aerosol particle administration to the subject's lung function, an optimal breathing pattern can be achieved. The user would no longer have to concentrate on the proper breathing technique because the present invention would induce the appropriate breathing pattern. This would inevitably improve user compliance and enable a precise, reproducible dosage of the pharmaceutical formulation to be delivered. The method may also comprise measuring at least one pulmonary function parameter of a subject.

In one aspect of the invention, the measured pulmonary function parameter is a measure of a subject's inspiratory function. Preferably, the inspiratory capacity (IC) of the subject is obtained. The inspiratory capacity is a measure of the maximal volume that the subject is capable of inhaling at a time. Using the subject's inhalation capacity, the appropriate inhalation volume and optimal aerosol parameters can be determined for the inhalation device or system. Preferably, the inhalation capacity is multiplied by a constant for determining the corresponding inhalation volume.

In another aspect of the invention, the inventors have unexpectedly found that, instead of requiring the subject's inspiratory capacity, expiratory function data can be used to sufficiently estimate the inspiratory capacity. As a measure of expiratory function, the pulmonary function parameter can be, for example, maximum expiratory flow (MEF), forced expiratory flow (FEF), forced expiratory vital capacity (FVC), forced expiratory volume (FEV), or forced expiratory volume per second (FEV₁).

In one embodiment, the measured expiratory function is the forced expiratory volume per sec (FEV₁). This parameter describes the maximal volume that a patient is capable of exhaling in one second. The inventors have found that the FEV₁ can be used as a correlation of the IC if the FEV₁ is corrected to consider the severity grade of the presence of a lung disease in the subject. Depending on the severity grade of the subject, a corrective coefficient can be calculated. The measured FEV₁ can be multiplied by a corrective coefficient to obtain a corrected FEV₁ parameter. The corrected FEV₁ parameter can then be used to determine the appropriate inhalation volume and aerosol parameters. This aspect of the invention has several advantages. Although inspiratory capacity has been generally used for estimating inhalation volumes, inspiratory capacity calculations are in many cases prone to error. FEV₁ measurements, on the other hand, are generally quite reliable and reproducible. Further, FEV₁ measurements are common and can be obtained even with hand-held spirometers.

With the methods of the present invention, the aerosol parameters can be adjusted to provide an optimal breathing pattern based on a subject's lung function data. The aerosol parameters include, but are not limited to, inhalation volume, the period between the number of breaths, inspiratory flow rate, the timing of the release of aerosol particles (or aerosol bolus) and/or of particle-free air.

In another aspect of the invention, aerosol parameter groups can be specified. Each aerosol parameter group can have predefined aerosol parameters. For example, each aerosol parameter group can have a different inhalation volume and/or different values for the aerosol parameters, such as aerosol bolus timing and inspiratory flow rate for example. The aerosol parameter group data can be stored in a memory means of the inhalation device or system, in a computer-readable medium, in an external storage device and/or transferred electronically to the inhalation device or system. In this aspect of the invention, the subject's lung function data is used to select the aerosol parameter group that enables the best breathing maneuver for the subject.

When the measured pulmonary function is a measure of the subject's inspiratory function, each aerosol parameter group has predetermined ranges for the measured inspiratory function parameter. In one embodiment, the aerosol parameters can be derived by comparing the value of the measured inspiratory function with predetermined ranges for prespecified aerosol parameter groups and by selecting the aerosol parameter group having a range, within which the measured inspiratory function parameter falls. Preferably, the measured inspiratory function parameter is the inspiratory capacity.

When the pulmonary function parameter is a measure of expiratory function, each aerosol parameter group has predetermined ranges for the corrected expiratory function. For example, the predetermined range values could be the same as those used for the case where inspiratory function data is considered. In one embodiment, the aerosol parameters are derived by selecting a corrective coefficient using the measured expiratory function from among a plurality of corrective coefficients derived from a standard, multiplying the measured expiratory function by the corrective coefficient to obtain a corrected pulmonary function parameter value, comparing the corrected pulmonary function parameter value with the predetermined ranges for the aerosol parameter groups, and selecting the aerosol parameter group having a range, within which the corrected pulmonary function parameter value falls.

The present invention is also directed to a computer readable medium comprising encoding means for implementing the methods of the present invention. The methods of the present invention are computer-implemented methods that can be carried out by a processor or controller of the inhalation device or system and/or by an external device.

The present invention is also directed to an inhalation system for administering aerosol particles to the lungs comprising a controller for adapting aerosol parameters for the inhalation system based on at least one measured pulmonary function parameter. The controller may comprise a processor using encoding means causing the system to implement the methods of the present invention. The system of the present invention can be designed to adapt the subject's inhalation maneuver automatically depending on the subject's lung function data. The system may also record and store the subject's breathing maneuvers, e.g., for compliance control. The system may also comprise a monitor for measuring at least one pulmonary function parameter of a subject.

The system of the present invention may be in any suitable form for an inhalation system. For example, the system of the present invention may be designed to receive a variety of detachable components, such as a mouthpiece, nebulizer or the like, and at least one cartridge or the like containing a pharmaceutical formulation. For example, the system of the present invention may comprise a mouthpiece connected in fluid communication with the inhalation flow path. The mouthpiece may be a permanent part of the housing or a detachable part.

The system of the present invention may comprise at least one orifice connectable to a source of aerosolized particles. The aerosolized particle source is preferably releasably or detachably connected to the device by any suitable means known in the art. The aerosolized particle source may be a powder dispersion device which utilizes a compressed gas to aerosolize a powder. The aerosolized particle source may be a nebulizer or the like, for aerosolizing solid or liquid particles. The nebulizer may be an ultrasonic nebulizer, a vibrating mesh nebulizer, a jet nebulizer or any other suitable nebulizer or vaporizer known in the field. These nebulizers can be separate components which can be attached to the system before use.

The system of the present invention may also comprise a reader for reading a memory means having a subject's individual parameters, lung function data and/or aerosol depositing parameters stored thereon. The memory means can be in the form of any computer readable storage medium known in the art, such as but limited to a storage stick, memory disk or electronic data card, such as a smart card. The reader can be in any form as known in the art. For example, the reader can be an interface or port, e.g. a USB port or the like for receiving a storage stick or a serial connection or drive for receiving a memory or electronic data card.

The system of the present invention may also comprise at lease one communication means for receiving and/or sending data associated with a subject's individual parameters, lung function data and/or aerosol depositing parameters. The communication means may be a wired connection or wireless connection sending and/or receiving data via infrared, microwave or radio frequency, optical techniques or any suitable manner known in the art. The communication means may be a telephone connection or jack. This would be advantageous if a health professional, e.g. a doctor, would like to adjust, from a remote location, the aerosol parameters based on lung function.

One embodiment of the present invention provides an inhalation system having aerosol parameters that can be adapted automatically depending on measurement of at least one lung function parameter indicative of a subject's inhalation or exhalation function to thereby tailor aerosol particle administration to the subject's breathing capabilities. Some examples of inhalation systems which can be used in conjunction with the methods and products of the present invention are described in WO 98/52633, EP 1 700 614 A1, European patent application no. 07 113 705.3 and European patent application no. 07 115 812.5, the entire contents of these documents being incorporated herein by reference.

The “aerosol particles” administered by the present invention may comprise at least one pharmaceutical formulation. The pharmaceutical formulations that may be aerosolized include powdered medicaments, liquid solutions or suspensions and the like and may include an active agent.

The term “pharmaceutical formulation” as used herein, includes active ingredients, drugs, medicaments, compounds, compositions, or mixtures of substances bringing about a pharmacological, often advantageous, effect. It includes food, food supplements, nutrients, medicaments, vaccines, vitamins, and other useful active ingredients. Moreover, the terms, as used herein, include any physiologically or pharmacologically active substances, bringing about a topical or systemic effect in a patient. The active ingredient lending itself to administration in the form of an aerosol can be an antibody, antiviral active ingredient, anti-epileptic, analgesic, anti-inflammatory active ingredient, and bronchodilator or can be an organic or inorganic compound, which without any restrictions can also be a medicament having an effect on the peripheral nervous system, adrenergic receptors, cholinergic receptors, skeletal muscles, cardiovascular system, unstriated muscles, circulatory system, neuronal connections, pulmonary system, respiratory system, endocrine and hormonic system, immune system, reproductive system, skeletal system, food supply system and excretory system, histamine cascade or central nervous system. Suitable active ingredients are for instance polysaccharides, steroids, hypnotics and sedatives, activators, tranquilizers, anticonvulsives (antispasmodics) and muscle35 relaxants, anti-Parkinson-substances, analgesics, anti-inflammatory agents, antimicrobial active ingredients, antimalarial agents, hormones, including contraceptives, symphatocomimetics, polypeptides and proteins producing physiological effects, diuretics, substances regulating the lipometabolism, anti-androgenic active ingredients, antiparasitics, neoplastic and antineoplastic agents, antidiabetics, food and food supplements, growth-promoters, fats, stool-regulators, electrolytes, vaccines and diagnostics.

The invention is particularly suited for inhalation application of different active ingredients, such as the following ones (without being restricted thereto): Insulin, calcitonin, erythropoietin (EPO), factor VII, factor IX, cylcosporin, granulozyte colony stimulating factor (GCSF), alpha-1-proteinase inhibitor, elcatonin, granulocyte macrophage colony stimulating factor (GMCSF), growth hormones, human growth hormone (HGH), growth hormone releasing hormone (GHRH), heparin, low molecular weight heparin (LMWH), interferon alpha, interferon beta, interferon gamma, interleukin-2, luteinizing hormone releasing hormone (LHRH), somatostatin, somatostatin-analogs, including octreotides, vasopressin analogs, follicle stimulating hormone (FSH), insulin-like growth factor, insulintropin, interleukin-I receptor antagonist, interleukin-3, interleukin-4, interleukin-6, macrophage colony stimulating factor (M-CSF), nerve growth factor, parathryoid hormone (PTH), thymosin alpha 1, IIb/IIIa inhibitor, alpha-1 antitrypsin, antibodies against respiratorily syncytic virus, cystic fibrosis transmembrane regulator gene (CFTR), desoxyribonuclease (Dnase), bactericides, permeability increasing protein (BPI), anti-CMV antibodies, interleukin-1-receptor, retinol, retinyl-ester, tocopherols and their esters, tocotrienols and their esters, carotinoids, in particular beta carotin and other natural and synthetic antioxidants, retinol acids, pentamides, albuterolsulfate, metaproterenolsulfate, beclomethasonedipropionate, triamcinolonacetamide, budesonidacetonides, ipratropium bromide, salbutamols, formanilides, flunisolides, fluticasones, cromolyn potassium, ergotamine tartrate and the analogs, agonists and antagonists of the above-mentioned substances.

Moreover, active ingredients can be nucleic acids in the form of pure nucleic acid molecules, viral vectors, associated viral particles, nucleic acids associated with or contained in lipids or a lipidcontaining material, plasmid DNA or plasmid RNA or other constructs from nucleic acids, which are suitable for cell transfection or cell transformation, in particular in the case of cells of the alveolar region of the lung.

The active ingredient can be present in different forms, such as soluble or insoluble, charged or uncharged molecules, components of molecular complexes or pharmacologically acceptable inactive ingredients. The active ingredient can be naturally occurring molecules or their recombinant products, or the molecules can be analogs of the naturally occurring or recombinantly produced active ingredients to which or from which one or more amino acids have been added or deleted. Moreover, the active ingredient can contain attenuated live vaccines or killed viruses for vaccination purposes. If the active ingredient is insulin, it includes naturally extracted human insulin, recombinant human insulin, insulin extracted from cattle and/or swine, recombinant porcine or bovine insulin and mixtures of the above-mentioned insulins. The insulin can be present in a substantially purified form, but can also contain usual commercial extracts. The term “insulin” also includes analogs, to which or from which one or more amino acids of the naturally occurring or recombinant insulin have been added or deleted.

In an experimental study, the methods of the present invention were used to determine aerosol parameters for patients diagnosed with lung disease, like chronic obstructive pulmonary disease (COPD) or asthma. The patient's lung function data was evaluated and used for allocating the patient to the best-suited aerosol parameter group. In this case, four aerosol parameter groups were set up, wherein each group was associated with certain aerosol parameters and inhalation volume. One of the purposes of the experimental study was to compare aerosol parameter group allocation achieved using inspiratory capacity data with aerosol parameter allocation achieved using forced expiratory volume data.

In a first aspect of the invention, each patient was allocated to an aerosol parameter group depending on the patient's inhalation capacity (IC) data. In the particular cases, the IC ranges used for the aerosol parameter groups are shown below in Table 1.

TABLE 1

| IC in [l] | Group | Inhalation Volume in [l] |
|---|---|---|
| 0.0-0.75 | excluded | 0.0 |
| 0.76-1.53 | 1 | 0.5 |
| 1.54-2.30 | 2 | 1.0 |
| 2.31-3.10 | 3 | 1.5 |
| higher than 3.10 | 4 | 2.0 |

The aerosol parameter group was determined by comparing the patient's IC with the preset IC ranges associated with the groups and selecting the group having a range, within which the patient's IC fits. For example, one patient in the study had an inhalation capacity of approximately 1.781. For that patient, the aerosol parameter group 2 was selected.

In a second aspect of the invention, each patient was allocated to an aerosol parameter group depending on the patient's forced expiratory volume per sec (FEV₁) data. The FEV₁ measurements were corrected using a corrective coefficient that was determined using the severity grade of the patient's lung disease, i.e. lung diseases like COPD or asthma GOLD standard.

The patients were classified into different categories depending on the severity of the disease. The classification for COPD i.e. is standard regulated by the Global initiative for chronic Obstructive Lung Disease and is commonly referred to as the GOLD class or standard. In the GOLD class, the classification of severity of obstructive lung disease presently includes four stages determined by spirometry measurements: stage I (mild) for FEV₁≧80% predicted (using appropriated normal values for the person's gender, age and height), stage II (moderate) for 50%≦FEV₁≦80% predicted; stage III (severe) for 30%≦FEV₁≦50% predicted; and stage IV (very severe) for FEV₁≦30% predicted. For each GOLD class, a corrective coefficient was calculated. These values are shown below in Table 2. FEV1_{gc} represents the corrected FEV₁ measurement. It should be noted that the various values for the GOLD classes can change yearly as they depend at least in part on experimental data.

TABLE 2

| Severity Grade | FEV1% of | Corrective | |
|---|---|---|---|
| (Gold class) | predicted | Coefficient (k) | Correction of FEV₁ |
| 1 | >80% | K1 ≈ 1.10 | FEV1_{gc} = FEV₁*k1 |
| 2 | 50%-80% | K2 ≈ 1.29 | FEV1_{gc} = FEV₁*k2 |
| 3 | 30%-50% | K3 ≈ 1.60 | FEV1_{gc} = FEV₁*k3 |
| 4 | <30% | K4 ≈ 2.42 | FEV1_{gc} = FEV₁*k4 |

Referring again to the patient with an inhalation capacity of 1.781, the patient had a measured FEV₁ of 1.471 which was 88% of the predicted value considering the patient's age, height and gender. As can be seen from Table 2, the patient would be classified in GOLD class 1. The GOLD class can be determined based on the measured FEV₁. The FEV1_{gc} parameter is obtained by multiplying the measured FEV₁ with the corrective coefficient. The corrective coefficient for GOLD class 1 is 1.10. In this case, FEV₁×k1 (1.47×1.10) yielded a FEV1_{gc} parameter of 1.62. As can be taken from Table 3 shown below, the patient would again be allocated to group 2.

TABLE 3

| FEV1_{gc} in [l] | Group | Inhalation Volume in [l] |
|---|---|---|
| 0.0-0.75 | excluded | 0.0 |
| 0.76-1.53 | 1 | 0.5 |
| 1.54-2.30 | 2 | 1.0 |
| 2.31-3.10 | 3 | 1.5 |
| higher than 3.10 | 4 | 2.0 |

In the majority of the cases, the patients were allocated to the same aerosol parameter groups irrespective of whether the IC data or the FEV₁ data was used. The experimental results suggest that the FEV₁ data could be reliably used, in place of the IC data for determining aerosol parameters.

### DESCRIPTION OF THE DRAWINGS

Some embodiments of the inhalation system of the invention will be described with reference to the following figures:

FIG. 1 is a schematic view of an embodiment of the system.

FIG. 2 is a schematic view of another embodiment of the system.

FIG. 3 is a schematic view of another embodiment of the system.

FIG. 4 is a perspective view of another embodiment of the system.

### DETAILED DESCRIPTION OF PREFERRED EMBODIMENTS

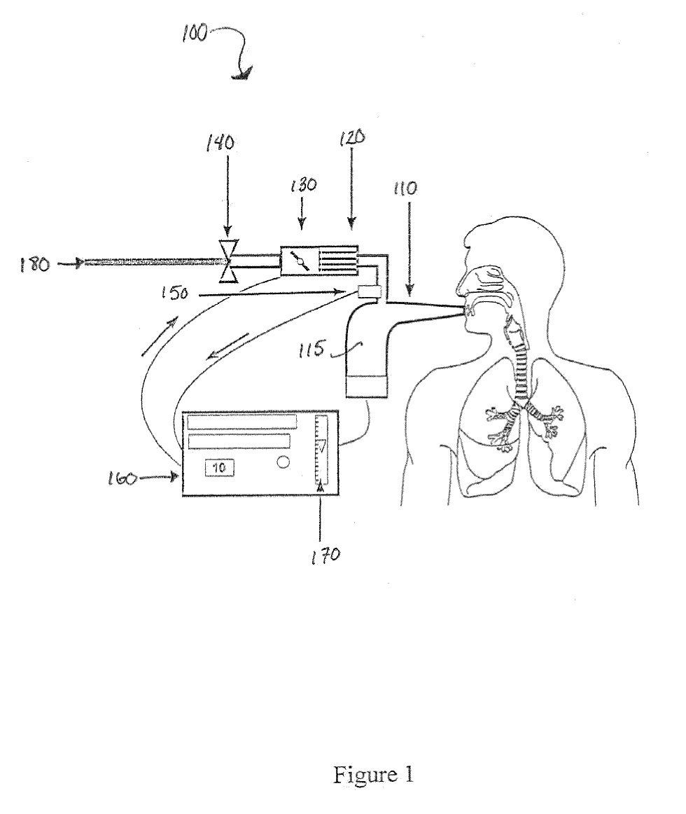

FIG. 1 illustrates a schematic view of an embodiment of a system which can be used for carrying out the methods of the present invention. Such a system is disclosed in WO 98/52633, the entire contents of which are incorporated by reference. System 100 for the administration of a medicated aerosol through the lung comprises an inhalation mouthpiece 110 with an associated vaporiser 115 which can be adjusted in terms of its operating phases as well as intensity/frequency. A volumetric flow controller 120, a compressed-air control valve 130, which is preferably configured as solenoid valve, a pressure reducer 140 and a compressed-air inlet 180 are disposed to be in flow communication with the inhalation mouthpiece 110. System 100 may also comprise a pressure sensor 150 which is responsive to a suction pressure in the mouthpiece for triggering the beginning of the vaporising operation of the vaporiser 115.

An electronic controller 160 is functionally connected to the compressed-air control valve 130, the pressure sensor 150 and the vaporiser 115. The electronic controller 160 is schematically represented as housing block which is additionally provided with an optical display of a flow meter 170 for checking the inhalation flow, for instance over a range of values from 0 to 1000 cm³/s. The volumetric flow controller 120 serves to maintain the inhalation flow constant over a particular range, for example from 0 to 1000 cm³/s. The compressed-air valve 130 is preferably designed as solenoid valve which switches the air supply.

Moreover, the inhalation period, the pause interval and the number of breathing cycles can be set on the electronic controller 160 in a manner not illustrated here, with a light-emitting diode being provided to issue a pause signal. For example, the individual functions which can be set, such as vaporising period or inhalation time, the pause interval and the ultrasonic vaporiser control, can be mentioned in text form in a block of the electronic controller 160, with a breathing cycle counter being included for detection of the breathing cycles.

In system 100, the nominal flow of inhalation can be initially controlled by means of flow meter 170, which includes a floating body, and then set to the desired amount at the volumetric flow controller 120 which maintains the inhalation flow constant. Then, the desired inhalation period can be set, e.g. using an input means of the electronic controller 160, within a particular range, for example from 0 to 20 seconds. The inhaled volume is then derived from the inhalation period and the inhalation flow. The desired duration of the pause interval can be equally set to be within a range, for example from 9 to 20 seconds. Additionally, the breathing cycle counter can be set to zero.

For instance, following these preparations, inhalation can now be performed in a way that the patient inhales at mouthpiece 110, which causes the pressure sensor 150 to respond and start inhalation automatically. The vaporiser 115 is supplied with compressed air throughout the pre-selected inhalation period, and the desired medicament is discharged in the form of a medicated aerosol from the mouthpiece 110 at a pre-selected flow rate. Upon expiration of the inhalation period, the compressed-air supply can be interrupted so that the subject cannot continue inhalation. The light-emitting diode signals to the subject that he or she should hold his or her breath. As soon as the pause interval has elapsed the pause interval LED can be extinguished, the patient exhales and the breathing cycle counter is incremented.

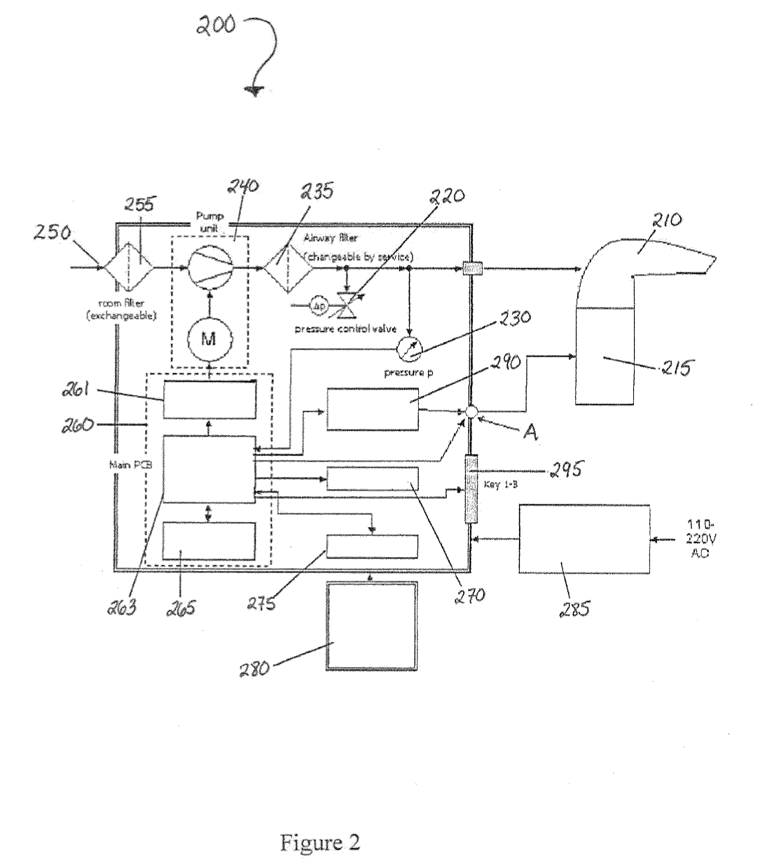

FIG. 2 schematically illustrates another embodiment of a system which can be used with the methods of the present invention. Such a system is described in EP 1 700 614 A1, the entire contents of which are incorporated by reference. System 200 for the application of a pharmaceutical aerosol via the lung comprises an inhalation mouthpiece 210 with an associated nebulizer 215. A pressure control valve 220, a pressure sensor 230, an exchangeable filter 235 (or pressure attenuator), a (pressure) air pump 240, a further exchangeable filter 255, and an air inlet 250 are operably connected to the inhalation mouthpiece 210. The filter 255 is assigned to the air inlet 250 in order to filter the air supplied to the air pump 240. The filter 235, the pressure sensor 230 and the pressure control valve 220 are arranged downstream from the air pump 240. The pressure sensor 230 is responsive to suction pressure in the mouthpiece 210 to trigger the nebulization start of the nebulizer 215.

System 200 may comprise control unit 260 having a motor controller 261, a microprocessor 263 and a memory 265. The control unit 260 may be functionally connected to the pump 240, the pressure sensor 230 and to the nebulizer 215 via corresponding nebulizer electronics 290 (e.g., a printed circuit board). The nebulizer electronics are preferably exchangeable so that the corresponding electronics can be used for the respective nebulizers. The nebulizer is connectable to the inhalation component via a connecting point A. Preferably, the connection A is an encoded connection, which thus enables an automatic recognition of the connected nebulizer. Thus, a plausibility test can be carried out, e.g., whether the pharmaceutical to be administered can be applied with the connected nebulizer at all.

The pressure sensor 230 can be used as feedback to the control unit 260 and serves to keep the inhalation flow constant in a particular range, for example from 0 to 1000 cm³/s.

In the control unit 260, the pharmaceutical and the inhalation volume can be set or adjusted via an input unit 295, e.g., via a keyboard or individual input keys. System 200 may also comprise a SmartCard device reader 275 via which individual inhalation parameters such as inhalation flow and inhalation volume stored on a SmartCard device 280 can be read in. The individual parameters can stored in the memory 265.

System 200 may also comprise an optical display unit 270 in order to show the subject the current inhalation flow. The display also serves to display the name of the pharmaceutical currently used, error messages, etc. The display preferably also serves to display the error messages of the nebulizer. System 200 may also comprise a power supply unit 285 (e.g., 110-240 V) for supplying current to the system.

The inhalation is carried out such that the patient inhales at the mouthpiece 210, whereby the pressure sensor 230 reacts and automatically starts the inhalation. The nebulizer 215 is supplied with compressed air via the air pump 240 during the inhalation, and the desired pharmaceutical in the form of aerosol emits with a pre-selected or predetermined flow from the mouthpiece 210. After expiry of the inhalation period and/or achievement of the inhalation volume, the air supply is interrupted and the patient cannot inhale further. The control unit 260 may control or activate the air pump 240 when the start of an inhalation process is recognized by the pressure sensor 230. When the inhalation process is terminated, the air pump 240 is deactivated again by the control unit 260.

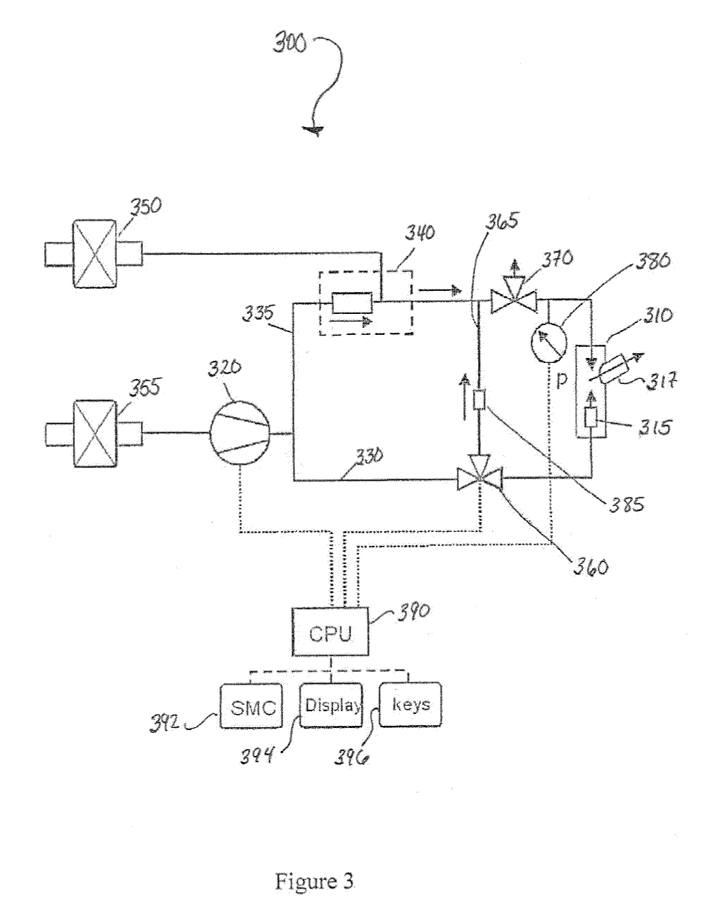

FIG. 3 shows a schematic view of another embodiment of a system which can used for the methods of the present invention. Such a system is disclosed in European patent application no. 07 115 815.5, the entire contents of which are incorporated herein by reference. System 300 comprises a compressor 320 for providing compressed air flow and a nebulizer 315 for providing aerosolized particle flow. The compressor 320 and the nebulizer 315 are connected to each other by means of an air channel 330. Further, system 300 comprises a mixer 310 for receiving the compressed air and/or the aerosolized particle flow and for providing at an outlet either a mixture of the compressed air flow and the aerosolized particle flow or only the compressed air flow or only the aerosolized particle flow. The mixer 310 may have an inhalation mouthpiece 317 provided at an outlet of the mixer 310. In a preferred embodiment of this system, the nebulizer 315 and mixer 310 are integrated as a single component as shown in FIG. 3. However, it is also possible to provide the nebulizer 315 and mixer 310 as separate components. In this case, the nebulizer and mixer can be connected using a channel.

The mixer 310 of system 300 is connected to the compressor 320 by an air channel 335. In this respect, the compressed air flow leaving the compressor 320 is separated into different flow paths; e.g., one flowing through air channel 330 and one flowing through air channel 335, as shown in FIG. 3.

System 300 may comprise a flow amplifier 340 as a means for increasing the compressed air flow. As shown in FIG. 3, the flow amplifier can be placed in fluid communication with air channel 335. In this respect, the flow amplifier is in fluid communication with mixer 310. The flow amplifier 340 can be in the form of venturi injectors 340. The venturi injectors use additional air flow, which is provided through air filter 350. The compressor 320 may also have an air filter 355 located at an inlet. The use of a flow amplifier provides several advantages. For one, the size of the inhalation system can be significantly reduced because a smaller compressor can be used. Also, with the use of a flow amplifier, it is no longer necessary to have a separate pressure-relief valve.

System 300 may also comprise a by-pass channel 365 that can be opened or closed using a by-pass valve 360. The by-pass channel 365 enables a constant inhalation flow even when aerosolizing is shut on or off. For example, this may be the case when aerosolizing is deactivated during the inhalation process. If the aerosolizing is interrupted during the inhalation process, the by-pass valve 360 is opened to redirect the compressed air flowing in air channel 330 from compressor 320 to by-pass channel 365. In this respect, the by-pass channel can be used to circumvent air flowing to the nebulizer 315. The by-pass channel 365 can be provided with an auxiliary injector 385 which functions in a similar manner as the nebulizer injector in nebulizer 315. The compressed air leaving the by-pass channel 365 and air channel 335 flows to mixer 310 such that a constant inhalation flow is provided to the patient even if the nebulizer is deactivated during the inhalation process. The deactivation of the nebulizer during inhalation may be advantageous for targeting aerosolized particles to particular areas of the lungs.

System 300 may also comprise a valve 370 for enabling excess air to be removed from the system, for example during an inhalation pause.

Further, system 300 may comprise a control unit 390 for controlling and/or regulating various components of the system, e.g., the valves and/or nebulizer. The control unit 390 can be configured to control the various components of the system according to a subject's individual aerosol and/or inhalation parameters. A pressure sensor 380 can be used for providing feedback to the control unit 390. The control unit 390 has a display 394 and input means, such as a keyboard, for entering information. System 300 may also comprise input means such as a reader 392 for reading an electronic storage means, such as a memory card (e.g. a smart card), a storage stick, storage disk or the like.

In FIG. 3, an exemplary system of this embodiment is shown. However, modifications to the system shown in FIG. 3 are possible and foreseen. For example, system 300 may comprise venturi injectors 340 without the presence of the by-pass channel 365 or may comprise a by-pass channel 365 without the presence of the venturi injectors 340.

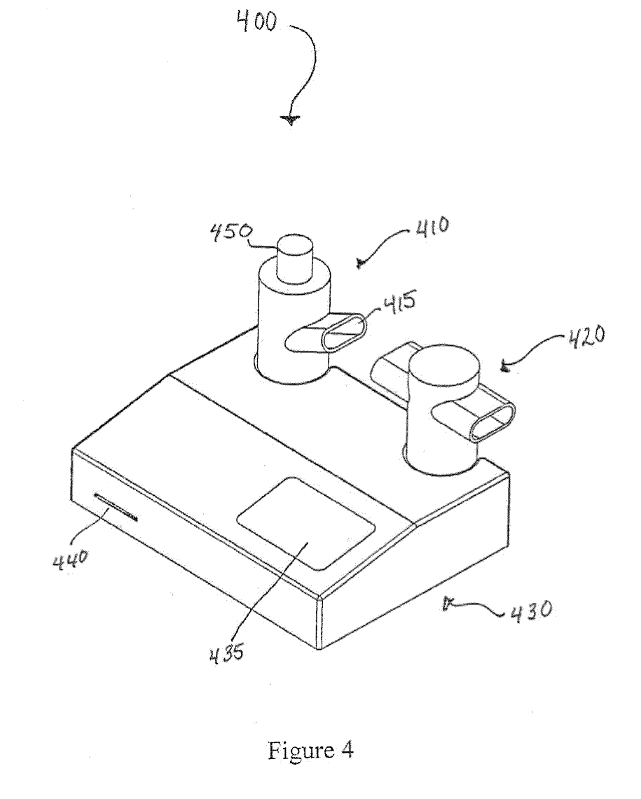

In FIG. 4, another embodiment of an inhalation system which can be used with the methods of the present invention is illustrated. Such a system is disclosed in European patent application 07 113 705.3, the entire contents of which are incorporated by reference. In this embodiment, the system 400 comprises an inhalation device 410, a spirometer 420 for measuring a lung function parameter and a base station 430 for receiving the device 410 and/or spirometer 420.

As depicted, the device 410 can be a hand-held, portable device. The device 410 has a housing with a mouthpiece 415. The mouthpiece 415 may be removed or replaced by a compatible mouthpiece. To this end, a connection is provided in the housing of device 410 for enabling a detachable connection with the mouthpiece 415. Alternatively, mouthpiece 415 may be an integral part of the housing of the device 410. Device 410 is also adapted to receive a cartridge or receptacle 450 holding the pharmaceutical formulation or drug. For example, the housing of device 410 can be manufactured such that cartridge 450 can simply be inserted into the top of the device as shown in FIG. 4.

The spirometer 420 can also be a hand-held, portable device as shown. Spirometer 420 may also have control buttons for controlling the operations of the spirometer and/or a display for showing measured results and/or settings.

The base station 430 may include cradles, or the like, for holding the device 410 and spirometer 420. The base station 430 may also serve as a charger for recharging any batteries provided in device 10 and/or spirometer 420. To this end, the cradles may include an interface enabling an electronic connection with device 410 or monitor 420. The interface could also enable the transfer of data between the base station 430 and the device 410 or spirometer 420. As depicted, base station 430 may also have a display 435 for displaying any desired information or data, for example the status of the base station 430, device 410 and/or spirometer 420. Base station 430 may optionally include a slot 440 for receiving a memory card, e.g. a smart card, having data with a subject's aerosol parameters. In this respect, multiple users could use the base station 430 for adapting their inhalation devices 410. Base station 430 may also include an additional reader for reading a storage medium like a memory stick. Although not shown, base station 430 may include communication means for enabling wired or wireless telecommunication and/or data transfer to and from a remote location.

In FIG. 4, an exemplary system of this embodiment is shown. However, modifications to the illustrated system are possible and foreseen. For example, the base station 430 of system may be used for receiving an inhalation device having an integrated spirometer and an additional monitor for measuring a health parameter such as a cardio-monitor measuring heart beat rate for example. The inhalation device itself may also have communication means and/or a card reader, for example.

The various embodiments and experimental results presented in the specification are used for the sake of description and clarification of the invention, and thus should not be interpreted as limiting the scope of the invention as such. Moreover, the present invention is realized by the features of the claims and any obvious modifications thereof.

1. A method for determining aerosol particle administration to the lungs, the method comprising:
deriving aerosol parameters for an inhalation system based on at least one measured pulmonary function parameter to thereby adapt the aerosol particle administration to a subject's lung function.

2. The method of claim 1, further comprising measuring at least one pulmonary function parameter of the subject.

3. The method of claim 1, wherein, when the pulmonary function parameter is a measure of an inspiratory function, the deriving aerosol parameters step comprises:
comparing the value of the pulmonary function parameter with predetermined ranges for prespecified aerosol parameter groups, each aerosol parameter group having predefined aerosol parameters; and
selecting a prespecified aerosol parameter group based on the comparison of the value of the pulmonary function parameters with the predetermined ranges.

4. The method of claim 1, wherein, when the pulmonary function parameter is a measure of an expiratory function, said deriving aerosol parameters step comprises:
selecting a corrective coefficient using the measured expiratory function from among a plurality of corrective coefficients derived from a standard;
multiplying the measured expiratory function by the corrective coefficient to obtain a corrected pulmonary function parameter value;
comparing the corrected pulmonary function parameter value with predetermined ranges for prespecified aerosol parameter groups, each aerosol parameter group having predefined aerosol parameters; and
selecting a prespecified aerosol parameter group based on the comparison of the corrected pulmonary function parameter value with the predetermined ranges.

5. The method of claim 4, wherein the corrective coefficient is based on a severity grade of the subject's lung disease.

6. The method of claim 1, wherein the pulmonary function parameter is maximum expiratory flow, forced expiratory flow, forced expiratory vital capacity or forced expiratory volume, or forced expiratory volume per second.

7. The method of claim 2, wherein the pulmonary function parameter is maximum expiratory flow, forced expiratory flow, forced expiratory vital capacity, or forced expiratory volume, or forced expiratory volume per second.

8. The method of claim 4, wherein the pulmonary function parameter is maximum expiratory flow, forced expiratory flow, forced expiratory vital capacity, or forced expiratory volume, or forced expiratory volume per second.

9. The method of claim 5, wherein the pulmonary function parameter is maximum expiratory flow, forced expiratory flow, forced expiratory vital capacity, forced expiratory volume, or forced expiratory volume per second.

10. A computer readable medium comprising encoding means for implementing the method of claim 1.

11. An inhalation system for administering aerosol particles to the lungs comprising:
a controller for adapting aerosol parameters for the inhalation system based on at least one measured pulmonary function parameter.

12. The system of claim 11, wherein the controller uses encoding means causing the system to implement the method of claim 1.

13. The system of claim 11, wherein the system comprises a reader for reading the computer readable medium of claim 10 or for reading a memory means having a subject's individual data and aerosol parameters stored thereon, wherein the controller has a memory for storing the subject's individual data and aerosol parameters.

14. The system of claim 11, further comprising a monitor for measuring at least one pulmonary function parameter of a subject.

15. The system of claim 11, wherein the aerosol parameters comprise inspiratory volume of aerosol particles, inspiratory volume of aerosol-free particles, inspiratory flow rate, inspiratory timing of inspiratory flow of aerosol particles, or inspiratory timing of inspiratory flow of aerosol-free particles.

16. The system of claim 11, wherein the system is a portable, hand-held inhalation device.

17. The method of claim 3, wherein the inspiratory function is inspiratory capacity.
